(19) United States
(12) Patent Application Publication
(10) Pub. No.: US 2024/0272175 A1
(43) Pub. Date: Aug. 15, 2024

(54) DIAGNOSTIC MARKER FOR FUNCTIONAL GASTROINTESTINAL DISORDERS

(71) Applicants: The University of Newcastle, Callaghan (AU); The University of Queensland, St. Lucia (AU)

(72) Inventors: Simon KEELY, Callaghan (AU); Nick TALLEY, Callaghan (AU); Grace BURNS, Callaghan (AU); Mark MORRISON, St. Lucia (AU)

(73) Assignees: The University of Newcastle, Callaghan, NS (AU); The University of Queensland, St. Lucia (AU)

(21) Appl. No.: 18/568,232

(22) PCT Filed: Jun. 7, 2022
(86) PCT No.: PCT/AU2022/050556

§ 371 Date: Dec. 7, 2023

(30) Foreign Application Priority Data
Jun. 7, 2021 (AU) 2021901692

(51) Int. Cl. G01N 33/68

(52) U.S. Cl. CPC G01N 33/6854; G01N 2333/315; G01N 2469/20; G01N 2800/06; G01N 2800/52

(57) ABSTRACT

Provided herein are methods for diagnosing functional gastrointestinal disorders such as functional dyspepsia and irritable bowel syndrome, comprising detecting IgG antibodies in a sample obtained from a subject, wherein the IgG antibodies recognise one or more antigens from Streptococcus salivarius. Also provided are kits for detecting IgG antibodies for use in the diagnosis of functional gastrointestinal disorders, and methods for selecting subjects for treatment for functional gastrointestinal disorders.

### FIELD OF THE ART

The present invention relates generally to immunological markers enabling or facilitating the diagnosis, or confirmation of diagnosis, of functional gastrointestinal disorders, in particular functional dyspepsia and irritable bowel syndrome.

### BACKGROUND

Functional gastrointestinal disorders are chronic disorders of unknown etiology, defined by patient symptoms and alteration to the function of the digestive system without obvious clinical pathology. The most common functional gastrointestinal disorders are functional dyspepsia and irritable bowel syndrome, estimated to affect between 12% to 30% of the general population worldwide.

Dyspepsia or indigestion, is one of the most common gastrointestinal disorders in both humans and animals. Dyspepsia refers to symptoms of persistent or recurrent abdominal pain or abdominal discomfort. Often dyspepsia is temporary and disappears spontaneously. However it can be a chronic and potentially debilitating condition. Chronic indigestion with no obvious cause, is referred to functional dyspepsia, or non-ulcer dyspepsia. Functional dyspepsia typically causes abdominal pain, bloating, gas and a feeling of fullness before finishing a meal.

Early studies focused on the stomach in functional dyspepsia. However in addition to its role as a major site of luminal antigen sampling, the duodenum has been demonstrated as a site of subtle inflammation. While eosinophils are a normal constituent in the gastrointestinal tract of healthy individuals, multiple studies have shown patients with functional dyspepsia to have greater numbers of duodenal eosinophils. Impaired integrity of the small intestinal barrier and increased mast cell numbers have also been shown in functional dyspepsia patients.

Irritable bowel syndrome is a common gastrointestinal disorder characterized by chronic abdominal pain, discomfort, bloating/distension and alteration of bowel habits in the absence of any detectable cause. The pathophysiology of irritable bowel syndrome remains unclear, yet studies have shown that numerous factors including alterations in gastrointestinal motility, visceral hypersensitivy, inflammation, cytokine release, alteration in fecal flora, and bacterial overgrowth may play a role.

Although studies into the duodenal mucosa-associated microbiota are in their infancy, various reports have demonstrated the most prevalent genera to be Streptococcus, Prevotella, Porphyromonas, Veillonella, Fusobacterium, and Neisseria, as well as members of the Paraprevotellaceae and Lactobacillaceae (see, e.g. Shanahan et al., 2016, Alimentary Pharmacology and Therapeutics 43:1186-1196). Little is known regarding the composition or function of the microbiota in functional gastrointestinal conditions. Increased bacterial load and reduced diversity in the duodenum of functional dyspepsia patients that could be correlated with meal related symptoms and poor quality of life scores have been reported, suggesting a role for the microbiota of the gastrointestinal tract in the pathophysiology of functional dyspepsia (see, e.g. Zhong et al., 2017, Gut 66:1168-1169). However the nature and consequence of the host-microbiota interactions and their relationship to symptoms of functional gastrointestinal disorders is not well understood.

Despite considerable research the etiology of functional dyspepsia and irritable bowel syndrome remains poorly understood. As such, efforts to diagnose, or confirm a diagnosis of, these disorders are hampered. Currently diagnosis of functional gastrointestinal disorders is typically based on patient described symptoms over time, defined by the Rome III or Rome IV criteria, coupled with negative endoscopic findings to rule out organic disease. Diagnosis is laborious, with undue stress on both the patient and the health system. In particular, endoscopy is invasive and costly. Identifying biomarkers for functional gastrointestinal disorders would accelerate the diagnosis of these conditions and be a significant saving to the healthcare system. In addition, while antibiotic treatment is successful for the treatment of some functional gastrointestinal disorder patients, efficacy is less than 50%, and there is no way to distinguish who will respond.

There is a clear need for the development of improved methods of diagnosing or confirming a diagnosis of functional gastrointestinal disorders such as functional dyspepsia and irritable bowel syndrome. Such diagnosis facilitates more effective treatment.

### SUMMARY OF THE INVENTION

The present invention is predicated on the inventors' identification of a dysregulation of the relationship between the duodenal microbiota and the immune system in functional dyspepsia patients. As described herein, the inventors have surprisingly detected IgG antibodies in plasma that are associated with functional dyspepsia, and directed against antigens from a bacterium isolated from the human duodenum and taxonomically affiliated with Streptococcus salivarius, hereafter referred to as strain AGIRA0003, deposited pursuant to the Budapest Treaty with the National Measurement Institute (Australia) on 28 Apr. 2021 under Accession Number V21/008005.

The identification of patients with bacterial/immune basis for functional gastrointestinal disorder represents a novel and powerful screening tool to identify patients responsive to antibiotics, thus reducing unnecessary therapies.

Accordingly, a first aspect of the present invention provides a method for diagnosing a functional gastrointestinal disorder in a subject, the method comprising detecting IgG antibodies in a sample obtained from the subject, wherein the IgG antibodies recognise one or more antigens from Streptococcus salivarius.

Typically the functional gastrointestinal disorder is functional dyspepsia or irritable bowel syndrome. In an exemplary embodiment the functional gastrointestinal disorder is functional dyspepsia.

In particular embodiments, the sample is a blood sample, typically a plasma sample.

Typically the Streptococcus salivarius is S. salivarius AGIRA0003 deposited pursuant to the Budapest Treaty on 28 Apr. 2021 under Accession Number V21/008005.

In particular embodiments, the Streptococcus salivarius is S. salivarius AGIRA0003 and the one or more antigens are selected from Group B Streptococcal (GBS) Bsp-like repeat protein, 30S ribosomal protein S2, and antigenic peptides or fragments thereof.

In an exemplary embodiment, the IgG antibodies are detected using an enzyme-linked immunosorbent assay (ELISA). Optionally, the method comprises contacting the sample with GBS Bsp-like repeat protein or an antigenic or immunoreactive peptide or fragment thereof, and/or 30S ribosomal protein S2 or an antigenic or immunoreactive peptide or fragment thereof.

Said detection of the IgG antibodies according to the method may be qualitative or quantitative. Optionally the method comprises comparing a determined level of the IgG antibodies in the sample to a reference or control value, wherein the reference of control value represents the level of the IgG antibodies in the absence of a functional gastrointestinal disorder. The reference or control value may be derived from one or more individuals known not to have a functional gastrointestinal disorder.

Another aspect of the present invention provides a kit for detecting IgG antibodies for use in the diagnosis of a functional gastrointestinal disorder, the kit comprising one or more antigenic or immunoreactive proteins, peptides or fragments selected from GBS Bsp-like repeat protein or an antigenic or immunoreactive peptide or fragment thereof, and/or 30S ribosomal protein S2 or an antigenic or immunoreactive peptide or fragment thereof, wherein said IgG antibodies recognise one or more antigens from Streptococcus salivarius.

Another aspect of the present invention provides a method for selecting a subject for treatment for a functional gastrointestinal disorder, comprising:
i) executing the step of detecting IgG antibodies in samples obtained from one or more subjects, and optionally determining the level of said antibodies to thereby diagnose the functional gastrointestinal disorder, wherein said IgG antibodies recognise one or more antigens from Streptococcus salivarius; and
ii) selecting a subject for treatment for the functional gastrointestinal disorder based on said IgG antibody detection or levels.

Another aspect of the present invention provides a method for monitoring the response of a subject to a treatment regime for a functional gastrointestinal disorder, or for evaluating the efficacy of a treatment regime in a subject with a functional gastrointestinal disorder, the method comprising:
i) treating the subject with a treatment regime for the functional gastrointestinal disorder for a period sufficient to evaluate the efficacy of the regime;
ii) obtaining a sample from the subject and detecting IgG antibodies in the sample, optionally determining the level of said antibodies, wherein said IgG antibodies recognise one or more antigens from Streptococcus salivarius;
iii) repeating step ii) at least once over a period of time while the subject is administered said treatment regime; and
iv) determining whether the level of said IgG antibodies change over the period of time, to thereby determine the response of the subject to the treatment regime and/or the efficacy of the treatment regime.

### BRIEF DESCRIPTION OF THE DRAWINGS

FIG. 1: Exemplary initial screening of Streptococcus and Corynebacterium microbial strains altered in functional dyspepsia patients for seroreactivity to patient plasma. Total protein extracted from bacterial lysates was electrophoresed and immunoblotted with patient plasma as the probing antibody to determine if there was evidence of interaction between the duodenal microbiota and patient plasma in functional dyspepsia (FD). (A, B) Representative immunoblots of this screening process. (C) The presence of such an interaction with either Streptococcus salivarius strain AGIRA0003 or strain 57.1 was tested for potential associations with FD in the screening population. n=6 controls n=15 FD. Statistical analysis: Chi-Squared test.

FIG. 2: Five clinical isolates assigned to Veillonella spp. were screened for seroreactivity in controls and functional dyspepsia patients. (A, B) representative blots demonstrating screening of controls and FD patient sera against 5 clinical isolates of Veillonella spp. recovered from human duodenal biopsies. (C) Chi-square testing for associations of the presence of seroreactivity against Veillonella atypica strain N227-1, (D) Veillonella atypica strain N221-1, (E) Veillonella strain atypica N221-2, (F)Veillonella atypica strain N221-3, (G) and Veillonella dispar strain N221-4 and a participant's status as control or FD patient. n=10 controls, n=15 FD patients. Species designations are bases on current bacterial taxonomy. Statistical analysis: Chi-square test, p=ns for all comparisons.

FIG. 3: The presence of Streptococcus salivarius AGIRA0003 IgG seroreactivity in FD patients when compared to controls, as demonstrated by (A) banding patterns observed in immunoblots where total protein extracted from the bacteria was probed with patient plasma. (B) The number of FD patients demonstrating a seroreactive response at any molecular weight was compared to seroreactive controls. The number of FD patients with a banding pattern located (C) between 100-75 kDa and (D) between 30-35 kDa was compared to the number of positive controls to determine if there was a relationship between this reaction and FD. n=17 controls, n=40 FD patients. Statistical analysis, Chi-square test, ***p<0.001 ****p<0.0001.

FIG. 4: Screening of Streptococcus salivarus AGIRA0003 total protein lysates for antibodies in plasma of controls and functional dyspepsia patients. Patients were screened for (A) IgM and (B) the proportions of IgM seroreactive FD patients and controls were analysed. (C) Patient plasma was screened for IgA antibodies against proteins from Streptococcus salivarius AGIRA0003 and (D) tested for associations with FD or controls. (E) No IgE response was detected in any of the controls or patients screened. n=6 controls and n=13 FD for (B), n=17 controls and n=39 FD for (D) and n=8 controls and n=21 FD for (E). Statistical analysis: Chi-square test.

FIG. 5: IgG seroreactive status is not associated with eosinophil number in functional dyspepsia patients. (A) Eosinophils were counted as an average of 5 high powered fields in duodenal biopsies stained with haematoxylin and eosin. Scale bar=50 um, yellow arrows identify example eosinophils. Eosinophil numbers were compared between controls, FD patients with IgG sero-positivity (IgG+) or IgG sero-negativity (IgG-) at (B) 70-100 kDa and (C) 30-35 kDa. (D) Sero-negative vs sero-positive controls were compared with sero-negative and sero-positive FD patients at (D) 70-100 kDa and (E) 30-35 kDa. (F) Flow cytometry was used to examine gut homing T cell populations in controls and FD patients. These populations were examined in (G) controls, FD patients with IgG sero-positivity (IgG+) or IgG sero-negativity (IgG-), and (H) the sero-positive and sero-negative control populations were also investigated. n=15 controls, n=36 FD for eosinophil counts, n=15 controls, n=33 FD for gut homing T cell analysis. Data presented as mean±SEM. Statistical analysis: Kruskal-Wallis test with Dunn's multiple comparisons test, *p<0.05.

FIG. 6: Identification of seroreactive proteins in Streptococcus salivarius AGIRA0003. The protein sequences obtained using mass spectrometry were blasted against the protein sequence determined from clinical isolates of Streptococcus salivarius AGIRA0003 to identify GBS Bsp-like repeat proteins as the proteins against which FD patients had IgG antibodies directed against. These proteins are in close proximity to a GH25 muramidase catalytic module.

FIG. 7: Immunoprecipitation products of human plasma IgG (FD patient) incubated with AGIRA0003 total protein lysates. Immunoblot of immunoprecipitation products probed with FD patient #1 plasma. Well 1—Marker, 2, AGIRA0003 total protein lysate, 3—FD patient #1 non-crosslinked IgG incubated with AGIRA0003 proteins, 4—same patient, crosslinked IgG incubated with AGIRA0003 proteins, 5—marker, 6—marker, 7—AGIRA0003 total protein lysate, 8—FD patient #2 non-crosslinked IgG incubated with AGIRA0003 proteins, 9—same patient, crosslinked IgG incubated with AGIRA0003 proteins, 10—marker. Boxes indicate rough area excised for mass spectromtetry analysis. M—light imaged marker.

The present specification contains amino acid and nucleotide sequence information prepared using the programme Patentln Version 3.5, presented herein in a Sequence Listing. The amino acid sequences of the Streptococcus salivarius seroreactive proteins identified in the current study are provided in SEQ ID NOs:1 and 2 herein.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein, the term “subject” may be used interchangeably with the term “individual” or “participant”. A “subject” may include any mammal, such as humans, non-human primates, livestock animals (eg. sheep, pigs, cattle, horses, donkeys, goats), laboratory test animals (eg. mice, rabbits, rats, guinea pigs, other rodents), companion animals (eg. dogs, cats). In preferred embodiments, the subject is a human.

The term “sample” as used herein includes any biological specimen obtained from a subject.

As used herein the term “antigen” means a substance that induces an immune response in a subject. The antigen may comprise, for example, a whole organism, a subunit or portion of an organism, a peptide, polypeptide or protein, polypeptide, a nucmelic acid molecule, or any combination thereof.

The terms “protein”, “polypeptide”, and “peptide” are used interchangeably herein to refer to polymers of amino acid residues of any length. As such the term “protein” encompasses not only the peptide or polypeptide product of a gene, but also functionally equivalent fragments, derivatives and variants thereof and post-translationally modified forms of the peptide or polypeptide product. Variants and derivatives typically exhibit at least some of the functional activity of the gene of which it is a variant or derivative. Different isoforms of a protein are also encompassed by this general term. Also encompassed by the term “protein” as used herein are mature protein and polypeptide sequences, in addition to proproteins, preproproteins and other precursor molecules including, for example, signal peptides, activation peptides or other sequences cleaved from a precursor molecule to generate the mature protein or polypeptide sequence. The protein, polypeptide or peptide can be linear or branched, it may comprise modified amino acids or amino acid analogs, and it may be interrupted by chemical moieties other than amino acids. The terms also encompass proteins, polypeptides and peptides that have been modified naturally or by intervention, for example by disulfide bond formation, glycosylation, lipidation, acetylation, phosphorylation, or any other manipulation or modification, such as conjugation with a labeling or bioactive component. Thus, as contemplated herein, proteins, polypeptides and peptides employed in the diagnostic methods of the invention may be recombinant proteins, polypeptides and peptides.

As used herein the term “treatment” refers to any and all treatments that remedy a condition or one or more symptoms of a condition or disease, prevent the establishment of a condition or disease, or otherwise prevent, hinder, retard, or reverse the progression of a condition or disease or other undesirable symptoms in any way whatsoever. Thus the term “treatment” is to be considered in its broadest context. For example, treatment does not necessarily imply that a patient is treated until total recovery.

In the context of functional gastrointestinal disorders, the relationship between the host immune system and resident microbes of the duodenal mucosa associated microbiome has remained largely unexplored. As exemplified herein, the present inventors demonstrate the presence of IgG antibodies in patient plasma directed against two proteins of Streptococcus salivarius strain AGIRA0003, isolated from the second portion of the duodenum in functional dyspepsia patients; one cell wall protein, Group B Streptococcal (GBS) Bsp-like repeat protein; and the ribosome-associated protein 30S ribosomal protein S2. The presence of these antibodies is significantly associated with functional dyspepsia.

Streptococcus salivarius is comprised of strains of anaerobic, Gram-positive bacteria that have been shown to colonise the oral cavity, jejunum, ileum, colon and upper respiratory tract, as well as human blood. S. salivarius strains can also be involved in biofilm formation, and have been implicated in the maintenance of homeostasis in the upper gastrointestinal tract through inhibition of the NFkB inflammatory pathway. S. salivarius has one of the largest numbers of cell wall associated and extra-cellular proteins (eg. adhesin proteins) in the salivarius group of Streptococci (S. salivarius, S. vestibularis, S. thermophilus) suggesting an advantage in colonisation and capacity to evade the host immune response.

As exemplified herein, the present inventors have identified a specific relationship between the presence of IgG antibodies against Streptococcus salivarius strain AGIRA0003 and functional dyspepsia, and have used gel electrophoresis, immunoblotting and mass spectrometry to analyse peptide spectra against the whole genome sequence of Streptococcus salivarius strain AGIRA0003 to identify the antigenic proteins as GBS Bsp-repeat domain protein (75-100 kDa; SEQ ID NO:1) and 30s ribosomal subunit S2 protein (30-35 kDa; SEQ ID NO:2). The inventors have thus identified useful biomarkers of functional gastrointestinal disorders such as functional dyspepsia. Given the reported heterogeneity of symptoms in functional dyspepsia, the calculated sensitivity of 90% and the specificity of 64.71% for the presence of plasma IgG antibodies directed against one or both of the two Streptococcus salivarius AGIRA0003 proteins identified by mass spectrometry suggests these biomarkers is capable of accurately detecting subjects with functional dyspepsia from controls with no evidence of functional or organic gastrointestinal disease.

The findings described herein demonstrate the presence of an adaptive, immunoglobulin-mediated response, facilitating the development of novel, minimally invasive diagnostic assays and tests for functional gastrointestinal disorders, for example using recombinant proteins as the basis for an enzyme linked immunosorbent assay (ELISA).

One aspect of the present invention provides a method for diagnosing a functional gastrointestinal disorder in a subject, the method comprising detecting IgG antibodies in a sample obtained from the subject, wherein the IgG antibodies recognise one or more antigens from Streptococcus salivarius, typically S. salivarius strain AGIRA0003.

Streptococcus salivarius strain AGIRA0003 was deposited, pursuant to the Budapest Treaty, with the National Measurement Institute (NMI), Australia on 28 Apr. 2021, which deposit was accorded Accession Number V21/008005.

The antigens typically comprise seroreactive proteins from S. salivarius or antigenic peptides or fragments of such proteins. In particular embodiments the seroreactive protein comprise GBS Bsp-like repeat protein, or 30S ribosomal protein S2. An amino acid sequence of GBS Bsp-like repeat protein from S. salivarius AGIRA0003 (PROKKA_00585) is set forth in SEQ ID NO:1. An amino acid sequence of 30S ribosomal protein S2 from S. salivarius AGIRA0003 (PROKKA_01557) is set forth in SEQ ID NO:2. Accordingly, in embodiments of the present invention the seroreactive proteins which the IgG antibodies recognise may comprise the proteins comprising amino acid sequences set forth in any one of SEQ ID NOs:1 and 2, or variants of said amino acid sequences having at least about 70% sequence identity thereto. In one embodiment the seroreactive protein may comprise an amino acid sequence at least about 70%, 75%, 80%, 85%, 90%, 92%, 94%, 96%, 98% or 99% identical to SEQ ID NO:1. In another embodiment the seroreactive protein may comprise an amino acid sequence at least about 70%, 75%, 80%, 85%, 90%, 92%, 94%, 96%, 98% or 99% identical to SEQ ID NO:2. Amino acid identity can be determined using a variety of tools well known to the skilled addressee, including sequence alignment programs such as CLUSTALW.

The IgG antibodies to be detected may recognise antigenic peptides and fragments of the above-described seroreactive proteins. These antigenic peptides and fragments are peptides and fragments that are capable of evoking an immune response, specifically the production of IgG antibodies in a subject. Typically the peptide or fragment is such that it has substantially the same immunological activity as the protein from which it is derived. Thus, a peptide or fragment according to the present invention comprises or consists of at least one epitope or antigenic determinant.

Suitable samples for use in the present invention include, without limitation, whole blood, plasma, serum, saliva, urine, stool, sputum, and any other bodily fluid, or a tissue sample (i.e., biopsy) such as a small intestine or colon sample, and cellular extracts thereof. The biological sample may be obtained by any suitable method, which may be determined by a person skilled in the art. In typical embodiments, the sample is a blood, plasma, or serum sample. In exemplary embodiments, the sample is a plasma sample. One skilled in the art will appreciate that samples such as plasma samples can be diluted prior to the analysis of marker levels.

In accordance with the methods of the present invention the IgG antibodies may be detected, and optionally the levels of IgG antibodies determined, using any means known to those skilled in the art. For example, in accordance with embodiments of the present invention the level of IgG antibodies present in a sample from a subject, can be determined by contacting the sample with a seroreactive protein having an amino acid sequence of SEQ ID NO:1 or 2.

The sample may be contacted with a conservative variant of a protein having an amino acid sequence of SEQ ID NO:1 or 2. A conservative variant is a protein substitution having one or more conservative amino acid substitutions. A conservative substitution denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term conservative substitution also includes the use of a substituted amino acid in place of an unsubstituted parent amino acid. Exemplary techniques for generating such amino acid insertion, deletion or substitution modifications include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction. Such techniques will be well known to those skilled in the art.

The sample may be contacted with a variant a protein having an amino acid sequence of SEQ ID NO:1 or 2 having at least about 70% sequence identity thereto. For example the protein may comprise an amino acid sequence at least about 70%, 75%, 80%, 85%, 90%, 92%, 94%, 96%, 98% or 99% identical to SEQ ID NO:1. For example the protein may comprise an amino acid sequence at least about 70%, 75%, 80%, 85%, 90%, 92%, 94%, 96%, 98% or 99% identical to SEQ ID NO:2.

The sample may be contacted with an antigenic or immunoreactive peptide or fragment of a protein having an amino acid sequence of SEQ ID NO:1 or 2, a conservative variant thereof or a variant having at least about 70% sequence identity thereto. Suitable antigenic or immunoreactive peptides and fragments can be identified using any number of epitope mapping techniques, well known in the art (e.g., Epitope Mapping Protocols in Methods in Molecular Biology, Vol. 66, Glenn E. Morris, Ed., 1996). For example, linear epitopes may be determined by, for example, concurrently synthesizing large numbers of peptides on solid supports, the peptides corresponding to portions of the seroreactive protein molecule, and reacting the peptides with antibodies while the peptides are still attached to the supports. Such techniques are known in the art and described in, e.g., U.S. Pat. No. 4,708,871. Similarly, conformational epitopes are readily identified by determining spatial conformation of amino acids such as by, e.g., x-ray crystallography and 2-dimensional nuclear magnetic resonance.

Seroreactive proteins, or antigenic or immunoreactive peptides or fragments thereof, used in accordance with methods of the invention may be obtained using standard techniques of recombinant DNA and molecular biology that are well known to those skilled in the art, including purification form host cells or using phage display. Guidance may be obtained, for example, from standard texts such as Sambrook et al., Molecular Cloning: A Laboratory Manual, Cold Spring Harbor, New York, 1989 and Ausubel et al., Current Protocols in Molecular Biology, Greene Publ. Assoc. and Wiley-Intersciences, 1992. Alternatively the seroreactive proteins, or antigenic or immunoreactive peptides or fragments thereof, may be synthesised by standard methods of liquid or solid phase chemistry well known to those of ordinary skill in the art. For example such molecules may be synthesised following the solid phase chemistry procedures of Steward and Young (Steward, J. M. & Young, J. D., Solid Phase Peptide Synthesis. (2nd Edn.) Pierce Chemical Co., Illinois, USA (1984), or Howl (ed.) Peptide Synthesis and Applications, Methods in Molecular Biology (Volume 298), 2005.

In embodiments of the present invention detection of IgG antibodies in a sample may be achieved using an immunoassay, such as an enzyme-linked immunosorbent assay (ELISA), an immunohistochemical assay or a mass spectrometry based assay. In particular embodiments the assay is an ELISA. ELISA techniques are well known to those skilled in the art. Suitable ELISA kits are available from a variety of sources, such as eR&D Systems, Inc. (Minneapolis, MN), Neogen Corp. (Lexington, KY), Alpco Diagnostics (Salem, NH), Assay Designs, Inc. (Ann Arbor, MI), BD Biosciences Pharmingen (San Diego, CA), Invitrogen (Camarillo, CA), Calbiochem (San Diego, CA), CHEMICON international, Inc. (Temecuia, CA), Antigenix America Inc. (Huntington Station, NY), QIAGEN Inc. (Valencia, CA), Bio-Rad Laboratories, Inc. (Hercules, CA), Bender MedSystems Inc. (Burlingame, CA), Agdia Inc. (Elkhart, IN), American Research Products Inc. (Belmont, MA), Biomeda Corp. (Foster City, CA), BioVision, Inc. (Mountain View, CA), and Kamiya Biomedical Co. (Seattle, WA).

In accordance with methods of the present invention the presence of IgG antibodies to one or more antigens from S. salivarius in a sample from a subject may be predicative or indicative of the subject having a functional gastrointestinal disorder such as functional dyspepsia or irritable bowel syndrome. Alternatively an elevated level of such IgG antibodies in a sample from a subject relative to a control value, or relative to levels of the IgG antibodies in one or more control samples, may be predictive or indicative of the subject having a functional gastrointestinal disorder such as functional dyspepsia or irritable bowel syndrome.

The term “control value” as used herein refers to a value for IgG antibody levels from one or more biological samples from healthy individuals or groups of individuals. A “control value” may comprise the compilation of data from one or more, typically a population, of healthy individuals whose diagnosis as a “control” for the purposes of the present invention has been confirmed. That is, for the purposes of practicing embodiments of the present invention samples to be used as controls need not be specifically or immediately obtained for the purpose of comparison with the sample obtained from the subject under assessment. “Control values” may comprise the measured value or the value following statistical analysis as described herein. A “control sample” as used herein refers to a sample obtained from a healthy individual from which a control value may be obtained. In the present context, a “healthy individual” is one that is either confirmed not to have a functional gastrointestinal disorder, who is Rome III-negative or Rome IV-negative for irritable bowel syndrome, does not have chronic gastrointestinal symptoms, does not have any active infections, and/or does not have significant chronic medical conditions.

IgG levels in samples obtained from subjects in accordance with the present methods may also advantageously be compared to one or more reference samples or reference values, determined from one or more, typically a population, of individuals known to have a functional gastrointestinal disorder. In subjects to which methods disclosed herein are applied, a comparison of the IgG levels with those of one or more reference samples or reference values may assist in determining diagnosis.

In some embodiments, the methods of the present invention comprise applying statistical analysis to levels of IgG antibodies determined for a subject. Statistical techniques that may be employed are known to those skilled in the art and include, but are not limited to, meta-analysis, linear regression analysis, multiple regression analysis, and receiver operator curve (ROC) analysis. ROC analysis is used to determine a score that is diagnostic with the greatest sensitivity and specificity. The ‘squarer’ the look of the curve, the simpler the determination of a diagnostic level or score. The closer the area under the curve is to 1 also indicates a result with high sensitivity and specificity. Such statistical analyses may be applied using a suitable algorithm such as, for example, regression analysis algorithm.

Those skilled in the art will appreciate that while exemplified herein with reference to the detection in a sample of IgG antibodies against one or more antigens from Streptococcus salivarius, diagnosing a functional gastrointestinal disorder such as functional dyspepsia or irritable bowel syndrome may also be achieved by detecting S. salivarius strain AGIRA0003, or one or both of the antigenic proteins from S. salivarius strain AGIRA0003 described herein, the Group B Streptococcus (GBS) B Surface Protein (Bsp)-like repeat protein (exemplified by the sequence of SEQ ID NO:1) and the 30S ribosomal protein S2 (exemplified by the sequence of SEQ ID NO:2), or antigenic peptide fragments thereof. The AGIRA0003 strain, antigenic proteins, or antigenic peptide fragments thereof, may be detected in a biological sample by any means known to those skilled in the art, including using probes and primers for nucleic acid-based detection techniques.

Those skilled in the art will appreciate that methods of the present invention may be employed in conjunction with one or more additional diagnostic methods or tools for the detection of functional gastrointestinal disorders. Such additional diagnostic methods or tools include, for example, assessment in accordance with the Rome III or Rome IV criteria, endoscopy or other physical examination or assessment. Methods of the invention may also be conducted in conjunction with detection or quantification of one or more other markers of gastrointestinal inflammation, such as gut-homing T cells and eosinophils, by methods well known to those skilled in the art.

The present invention also provides kits suitable for use in accordance with the methods of the invention. Such kits include for example diagnostic kits for assaying biological samples, comprising an agent(s) for detecting and/or determining the levels of IgG antibodies as disclosed herein, and reagents useful for facilitating said detection and/or determination of antibody levels. Kits according to the present invention may also include other components required to conduct the methods of the present invention, such as buffers and/or diluents. The kits typically include containers for housing the various components and instructions for using the kit components in the methods of the present invention.

A subject identified, in accordance with the methods of the present invention described hereinbefore as having a functional gastrointestinal disorder, can be selected for treatment, or stratified into a treatment group, wherein an appropriate therapeutic regimen can be adopted or prescribed with a view to treating the condition.

Thus, in an embodiment, the methods disclosed herein may comprise the step of exposing (i.e., subjecting) a subject identified as having a functional gastrointestinal disorder to a therapeutic treatment or regimen for treating the condition. The nature of the therapeutic treatment or regimen to be employed can be determined by persons skilled in the art and will typically depend on factors such as, but not limited to, the age, weight and general health of the subject.

An aspect of the present invention therefore provides a method for selecting a subject for treatment for treatment for a functional gastrointestinal disorder, comprising:
i) executing the step of detecting IgG antibodies in samples obtained from one or more subjects, and optionally determining the level of said antibodies to thereby diagnose the functional gastrointestinal disorder, wherein said IgG antibodies recognise one or more antigens from Streptococcus salivarius; and
ii) selecting a subject for treatment for the functional gastrointestinal disorder based on said IgG antibody detection or levels.

It will be clear to the skilled addressee that the methods disclosed herein can be also used to monitor the response of a subject to, and the efficacy of, a treatment for a functional gastrointestinal disorder, whereby IgG antibodies as described herein may be detected, or levels determined, at two or more separate time points, optionally including before commencement of treatment, during the course of treatment and after cessation of treatment, to determine whether said treatment is effective.

Thus, the invention provides a method for monitoring the response of a subject to a treatment regime for a functional gastrointestinal disorder, or for evaluating the efficacy of a treatment regime in a subject with a functional gastrointestinal disorder, the method comprising:
i) treating the subject with a treatment regime for the functional gastrointestinal disorder for a period sufficient to evaluate the efficacy of the regime;
ii) obtaining a sample from the subject and detecting IgG antibodies in the sample, optionally determining the level of said antibodies, wherein said IgG antibodies recognise one or more antigens from Streptococcus salivarius;
iii) repeating step ii) at least once over a period of time while the subject is administered said treatment regime; and
iv) determining whether the level of said IgG antibodies change over the period of time, to thereby determine the response of the subject to the treatment regime and/or the efficacy of the treatment regime.

Where the method indicates that the treatment regime is ineffective and/or the subject is not responding sufficiently to the treatment (i.e. no or insignificant reduction in IgG antibody levels), the method may further comprise altering or otherwise modifying the treatment regime with a view to providing a more efficacious or aggressive treatment. This may comprise administering to the subject additional doses of the same agent with which they are being treated or changing the dose and/or type of medication or other treatment.

Thus, reliable diagnoses of functional gastrointestinal disorders, such as are possible utilising methods of the present invention, facilitate decision making with respect to the most appropriate intervention or treatment regime for individual subjects. For example, the methods disclosed herein may assist in determining the likelihood that a subject may benefit from a particular treatment. Exemplary treatments may include, for example, the administration of therapeutic agents such as antibiotics (e.g. rifaximin), proton pump inhibitors, phytotherapeutic drugs, serotonergic agents, antidepressants, chloride channel activators, chloride channel blockers, guanylate cyclase agonists, antibiotics, opioid agonists, neurokinin antagonists, antispasmodic or anticholinergic agents, belladonna alkaloids, barbiturates, GLP-1 analogs, CRF antagonists, or the administration of therapy such as psychotherapy or physical therapy.

The treatment regime may be tailored to the subject themselves based on the levels of IgG antibodies detected and/or one or more other factors such as the severity of symptoms, the subject's lifestyle, age, weight, general health etc. For example, this may comprise introducing a new treatment regime or modifying an existing regime employed by the subject. Such decision making with respect to treatment regimes will vary from case to case and the determination of the most appropriate strategy is well within the expertise and experience of those skilled in the art.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### Examples

The following examples are illustrative of the invention and should not be construed as limiting in any way the general nature of the invention of the description throughout this specification.

### Example 1—General Methods

### Participant Selection and Recruitment

Participants were recruited from outpatient gastroenterology clinics at the John Hunter, Gosford and Wyong hospitals in New South Wales, Australia. Using the Rome III criteria, patients were diagnosed with functional dyspepsia. Control participants were undergoing a screening endoscopy for iron deficiency anaemia, a positive faecal occult blood test or dysphagia. Exclusion criteria for study participants included: a BMI greater than 40 (class III obesity), insulin dependent diabetics and pregnant women. Participants completed a medical interview at time of endoscopy, as well as a validated outpatient questionnaire incorporating modified Rome III questions for diagnosis of irritable bowel syndrome and functional dyspepsia (Drossman, 2006, Gastroenterology 130:1377-1390). Participants also completed the modified Nepean Dyspepsia Index (Talley et al., 1999, Alimentary Pharmacology & Therapeutics 13:225-235). All work was carried out with approval from the Hunter New England Local Health District Ethics Committee (External HREC reference number 13/12/11/3.01).

### Sample Collection and Processing

During endoscopy, biopsies were collected from the second portion of the duodenum (D2) and immediately placed into either a volume of RNAlater (Sigma-Aldrich, St. Louis, Missouri, USA) or immersed in a sterile, anaerobically prepared solution of mineral salts and containing 15% (v/v) glycerol, and stored at −80° C. for characterisation of the duodenal microbiota. Additional D2 biopsies were collected in 10% neutral buffered formalin for histological processing. Approximately 36 ml of heparinised blood was collected from each participant. Density gradient centrifugation with Lymphoprep™ media (StemCell Technologies Inc, Vancouver, Canada) allowed for the isolation of peripheral blood mononuclear cells (PBMCs) and plasma, according to manufacturer's instructions. Isolated PBMCs were resuspended in freezing media (10% DMSO, 90% FCS) or in a 1:1 ratio of freezing media and complete Roswell Park Memorial Institute (RPMI)-1640 media (supplemented with 10% FCS, 1% HEPES, 1% L-glutamine, 1% sodium pyruvate, 0.2% penicillin-streptomycin) before storage in liquid nitrogen.

### Staining and Acquisition of Cells for Flow Cytometry

After thawing, cells were rested overnight at 37° C./5% CO₂. Cells were then incubated with a fixable viability dye (conjugated to AF700, BD Biosciences, Franklin Lakes, USA), followed by an Fc block antibody (BD Biosciences). Cells were stained with antibodies directed against CD3 (conjugated to BUV805, BD Biosciences), CD4 (conjugated to FITC, BD Biosciences), integrin alpha 4 (CD49d, conjugated to PE-CF594, BD Biosciences), integrin beta 7 (conjugated to BV650, BD Biosciences) and chemokine receptor 9 (CCR9 conjugated to APC, BD Biosciences) for 30 minutes at 4° C. A LSRFortessa™ X20 flow cytometer with FACSDiva software (BD Biosciences) was used to acquire cell profiles. FlowJo v.10 software (BD Biosciences) was used to analyse acquired data. The cell population of interest were CD4+gut-homing T cells (CD3+CD4+integrin c; +integrin 7+CCR9+).

### Identification of Eosinophil Number

Formalin fixed, paraffin embedded D2 biopsies were stained with haematoxylin and eosin. Slides were digitalised using the Aperio AT2 (Leica Biosystems, Wetzlar, Germany) and Aperio ImageScope software (Leica Biosystems) was to view sections. Eosinophils were quantified in five randomly selected fields and the mean eosinophil number per high power field was calculated for each participant, as previously described (Ronkainen et al., 2019, Alimentary Pharmacology & Therapeutics 50:24-32).

### Duodenal Microbiome Identification and Classification

Genomic DNA was extracted from D2 biopsies stored in RNAlater using a repeated bead beating and automated column-based purification protocol (Shanahan et al. 2018 Microbiome volume 6, Article number: 150). The extracted genomic DNA was then subsampled and used for PCR amplification of the V6-V8 hypervariable regions present within Bacterial/Archaeal 16S rRNA genes. These products were further modified and elongated to include oligonucleotide sample-specific barcodes (Shanahan et al. 2018 Microbiome volume 6, Article number: 150) and sequenced using the Illumina MiSeq platform and the standardised protocols of the Australian Centre for Ecogenomics (ACE, (www.ecogenomic.org).

Biopsy tissue stored in the sterile, anaerobically prepared cryopreservative buffer was aseptically transferred within an anaerobic chamber to a 10 ml volume of anaerobically prepared Brain-Heart Infusion (BHI) broth with added hemin and vitamin K. The culture tubes were then incubated at 37° C. overnight, returned to the anaerobic chamber, and a 0.1 ml volume of the resulting cultured bacteria was taken and used to stage a 10-fold serial dilution, with 0.1 ml aliquots from each dilution plated onto BHI agar medium with added hemin and vitamin K. Following the incubation of these plates within the anaerobic chamber at 37° C., discrete colonies were sampled with a sterile, disposable inoculation loop, and propagated on fresh agar plates as described above. Following visual and microscopic purity checks, a single colony was used to inoculate 10 ml fresh BHI broth with added hemin and vitamin K, cultured overnight at 37° C., and −3 ml was mixed with the cryopreservation buffer and stored at −80° C. for later use.

The remainder of the cultures (˜7 ml) for the selected bacterial isolates were subjected centrifugation to collect the microbial biomass, which was then resuspended with a minimal volume of sterile Ringer's solution. The genomic DNA from this biomass was extracted using the protocol outlined above, and subsamples were used to produce the libraries needed for the production of high quality draft genome sequence data with the Illumina NextSeq platform and standardised protocols at the Australian Centre for Ecogenomics. The data (150 bp paired-end sequence reads) were quality-filtered using Trimmomatic (version 0.36), subjected to de novo assembly using the SPAdes Genome Assembler (version 3.11.0) and annotated using PROKKA (version 1.12).

Protein Extraction from Bacterial Lysates

Pellets of bacterial isolates were resuspended in Radio ImmunoPrecipitation Assay (RIPA) buffer (Sigma-Aldrich) containing protease and phosphatase inhibitors (HALT cocktail, Thermo Fischer Scientific). Samples were incubated and then vortexed to lyse cells, and centrifuged to remove unbroken cells and/or large debris. The resulting soluble protein lysates were then aliquoted and stored at −20° C. A bicinchoninic acid (BCA) assay kit (Thermo Fischer Scientific) was used to determine the concentration of total protein in the bacterial lysates. The assay was performed according to the manufacturer's instructions and the absorbance of samples was measured at 550 nm and Soft Max Pro (Molecular Devices) software determined the standard curve. From the standard curve, the concentration of protein in the bacterial lysates was calculated.

### Immunoblotting for Seroreactive Immunoglobulin Antibodies

Bacterial lysates underwent separation based on molecular weight by sodium dodecyl sulfate (SDS) polyacrylamide gel electrophoresis (PAGE) under reducing and denaturing conditions. Every second well contained a protein marker to facilitate cutting of the PVDF membrane following blocking. Electrophoresis was conducted in SDS running buffer (lx tris-glycine with 0.1% SDS), before proteins were transferred to a polyvinylidene difluoride (PVDF) membrane. Following protein transfer, membranes were blocked for 1 hour to prevent non-specific antibody binding in 2.5% BSA/2.5% skim milk powder. Blots were cut into single well strips, guided by the protein marker for probing with individual samples. Plasma samples from functional dyspepsia patients and controls were used in place of a primary antibody to detect any antibodies directed against duodenal bacterial proteins, as adapted from Lodes et al. Plasma samples were diluted 1:500 in 1% BSA/1% skim milk powder and incubated at 4° C. overnight. Blots were then incubated for 2 hours with anti-human HRP secondary antibodies directed against IgG, IgM, IgA or IgE (Sigma-Aldrich) diluted 1:1000 in 2.5% BSA/2.5% skim milk powder. Images of immunoblots were captured with a ChemiDoc MP System (Bio-Rad, Hercules, USA) the presence or absence of banding was recorded for each patient and each control.

Isolation of Seroreactive Proteins from Total Bacterial Proteins for Identification by Mass Spectrometry

Electrophoresis of proteins from the bacterial species of interest and a sero-negative control target were conducted as described above. Two Identical gels were run in tandem per bacterial target, to allow one to undergo fixation and overnight staining with Sypro Ruby; while the other underwent immunoblotting, as described above. Both bacterial samples were probed with the same plasma sample. The image generated of the specific proteins of interest by immunoblots was overlayed on the total protein image taken from the corresponding Sypro Ruby stained gel. The protein bands of interest in the total protein gel were identified and excised using a scalpel blade. Gel plugs from the corresponding area were also collected from the sero-negative bacterial lysate gel and protein free areas of both gels were obtained for background subtraction during mass spectrometry. Gel plugs were de-stained, washed and dehydrated using 100% acetonitrile a vacuum centrifugation. Samples were prepared for mass spectrometry based on a previously described protocol (Shevchenko et al., 2006, Nature Protocols 1:2856-2860). Briefly, sequencing grade trypsin (Promega, Madison, Wisconsin, USA) and ammonium bicarbonate solution was used for the re-hydration and overnight peptide digestion of gel plugs. 10% trifluoroacetic acid was used to quench trypsin, and sample supernatants were collected and stored at −20° C. following sonication prior to analysis.

### Liquid Chromatography Tandem Mass Spectrometry (LC-MS/MS)

Mass spectrometry was performed using a Q-Exactive Plus hybrid quadrupole-Orbitrap MS system (Thermo Fisher Scientific, Bremen, DE) coupled to a Dionex Ultimate 3000RSLC nanoflow HPLC system. Samples were loaded onto an Acclaim PepMap100 C18 75 μm×20 mm trap column (Thermo Fisher Scientific) for pre-concentration and online desalting. Separation was then achieved over an EASY-Spray PepMap C18 75 m×250 mm column (Thermo Fisher Scientific, Bremen, DE), employing a linear gradient from 2% to 32% acetonitrile at 300 nl/min over 120 min.

Data Dependent Acquisition (DDA), was performed on the Q-Exactive Plus MS System operated in full MS/data-dependent MS/MS mode. A precursor ion of endogenous peptide was measured in the Orbitrap scanning the mass range from m/z 390-1400 with an Orbitrap resolution of 70,000, a target automatic gain control (AGC) value of 1e6, and maximum fill times of 50 ms. The 20 most intense multiply charged precursors were selected for higher-energy collision dissociation (HCD) fragmentation with a normalised collisional energy (NCE) of 30, the MS/MS fragments were measured at an Orbitrap resolution of 17,500 AGC of 5e5, and maximum fill times of 110 ms.

Identification of Seroreactive Proteins from Clinical Isolates of Streptococcus salivarius AGIRA0003

The individual tryptic peptide sequences obtained from the mass spectrometry analysis of the excised polypeptides from the 1DE immunoblot samples were manually compared to the PROKKA-generated protein coding sequences predicted from the genome sequence data of S. salivarius AGIRA0003. Multiple tryptic peptides recovered from (GB_1D_03_B1) were matched with the presumptive “GBS Bsp-repeat domain protein” coding sequence predicted from the PROKKA annotation of the AGIRA0003 genome. Additionally, multiple tryptic peptide masses produced from (GB_1D_03_B2) were matched with the presumptive “30S ribosomal subunit S2 protein” predicted from the PROKKA annotation of the AGIRA0003 genome. The complete coding sequence (CDS) for the S. salivarius AGIRA0003 “GBS Bsp-repeat domain protein” was then used as a query sequence for a BLASTp-based comparison against all non-redundant GenBank CDS translations+PDB+SwissProt+PIR+PRF (excluding environmental samples from WGS projects) database curated by the National Centre for Biotechnology Information

### Statistical Analysis

Datasets were analysed and graphed using Graphpad Prism 8 software version 8.1 (Graphpad Software Inc., La Jolla, USA). Data was analysed for normality of distribution using the D'Agostino & Pearson test, where p<0.05 indicated data that was not normally distributed. The mean demographic characteristics of the functional dyspepsia patients versus controls were analysed by parametric t tests where the data was normally distributed and by the equivalent non-parametric t test where the distribution was not normal. This cohort data is presented as mean±SD and p<0.05 is considered significant. Fischer's exact test was used to analyse potential effects of co-morbidities and confounders between the control cohort and functional dyspepsia patients, as well as between functional dyspepsia patients identified as seropositive and those identified as seronegative. This data is presented as percentage of total cohort positive for tested variable and p<0.05 is considered significant.

Relationships between the presence or absence of a seroreactive response between functional dyspepsia patients and controls were analysed by Chi-square testing. This data is presented as percentage of total cohort positive for tested variable and p<0.05 is considered significant. Potential associations between seroreactivity status and other immune parameters were evaluated by ordinary one-way ANOVA for normally distributed datasets, while non-parametric ANOVA (Kruskal-Wallis test) with Dunn's multiple comparisons testing was used for data without a normal distribution. This cohort data is presented as mean±SEM and p<0.05 is considered significant.

### Example 2—Demographic and Diagnostic Characteristics of Study Cohort

A total of 17 controls and 40 functional dyspepsia (FD) patients were included in this study (Table 1). According to the Rome III criteria, 13 of the functional dyspepsia patients had post-prandial distress syndrome (PDS), 8 met the criteria for epigastric pain syndrome (EPS) and 19 reported symptoms of both EPS and PDS (EPS/PDS). The control population consisted of individuals referred to endoscopy for symptoms of dysphagia (n=6), unexplained iron deficiency anaemia (IDA) (n=8), unexplained reflux (n=1) or a positive faecal occult blood test (+FOBT) (n=2). No evidence of organic gastrointestinal disease was found during endoscopy or reported following clinical histological examination for this study cohort.

TABLE 1
DEMOGRAPHIC AND CLINICAL CHARACTERISTICS OF STUDY COHORT

| | Controls | | FD | | |
|---|---|---|---|---|---|
| | n = 17 | | n = 40 | | p value |
| Age (mean ± SD) | 53.59 | (11.71) | 46.45 | (17.42) | 0.152 |
| Female (%) | 10 | (58.82) | 33 | (82.50) | 0.091 |
| BMI (mean ± SD) | 27.18 | (5.83) | 27.31 | (5.65) | 0.738 |
| PPI use (%)# | 2 | (14.29) | 12 | (42.86) | 0.087 |
| H2RA use (%)# | 0 | | 4 | (14.29) | 0.280 |
| NSAIDS use (%)# | 0 | | 3 | (10.71) | 0.541 |
| Helicobacter pylori positive (%)# | 2 | (18.18) | 1 | (3.57) | 0.187 |
| IBS co-morbidity (%)# | 1 | (5.88) | 14 | (35.00) | 0.025* |

#denotes a factor that was not provided by all participants included in this cohort
BMI = body mass index, PPI = proton pump inhibitor, H2RA = H2 receptor antagonist, NSAIDS = non-steroidal anti-inflammatory drugs, IBS = irritable bowel syndrome

The mean age was 53.59±11.71 for controls and 46.45±17.42 for functional dyspepsia patients. 58.82% of the control cohort were female, compared to 82.50% of the functional dyspepsia patients, and there was no significant difference in body mass index (BMI) between controls (27.18±5.83) and functional dyspepsia patients (27.31±5.65). 18.18% of the control population tested positive for Helicobacter pylori infection, compared to 3.57% of functional dyspepsia patients. The rate of proton pump inhibitor (PPI) use was 42.86% in the functional dyspepsia cohort compared to 14.29% in controls (p=0.087) and there was no difference between histamine2-receptor antagonist (H2RA) or non-steroidal anti-inflammatory drug (NSAID) use between controls and functional dyspepsia patients (Table 1). The proportion of functional dyspepsia patients with co-morbid IBS was significantly higher than the controls (5.88% vs 35.00%, p=0.025).

### Example 3—Interactions Between the Immune Response and Microbiota in Functional Dyspepsia Patients

To investigate potential interactions of the immune system and microbiota in functional dyspepsia, immunoblotting was used to screen eight bacterial strains isolated from the duodenal biopsies collected from a human subject undergoing gastroscopy, against plasma from both FD patients and controls. The species chosen for screening were: Streptococcus salivarius strain 57.1, Streptococcus gordonii strain Challis CH1, Streptococcus salivarius strain AGIRA0003, a strain affiliated with Corynebacterium argentoratense DSM44202, Veillonella atypica strain N227-1, Veillonella atypica strain N221-1, Veillonella atypica strain N221-2, Veillonella atypica strain N221-3 and Veillonella dispar strain N221-4.

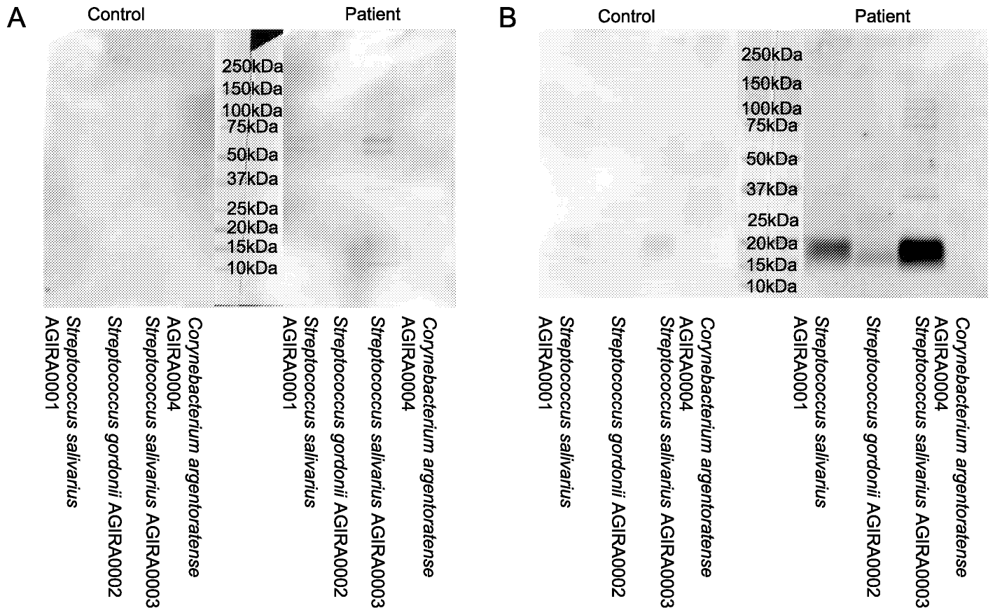

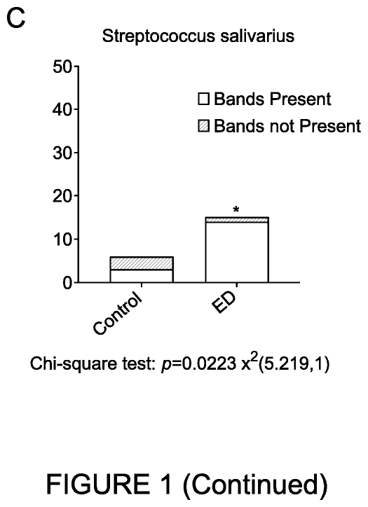

The results of initially screening 6 controls and 15 functional dyspepsia patients against Streptococcus salivarius 57.1, Streptococcus gordonii Challis CH1, Streptococcus salivarius AGIRA0003 and the strain affiliated with Corynebacterium argentoratense DSM 44202 (FIG. 1A, B) indicated the presence of bands at approximated 70-100 kDa and 30-35 kDa in 3 (50%) controls and 14 (93.33%) functional dyspepsia patients in both strains of Streptococcus salivarius screened. The band intensity for Streptococcus salivarius AGIRA0003 was visually greater than the intensity of banding for Streptococcus salivarius 57.1. The presence of any IgG seroreactive banding for either strain was significantly associated with a diagnosis of functional dyspepsia (χ²(5.219, 1), p=0.022) (FIG. 1C).

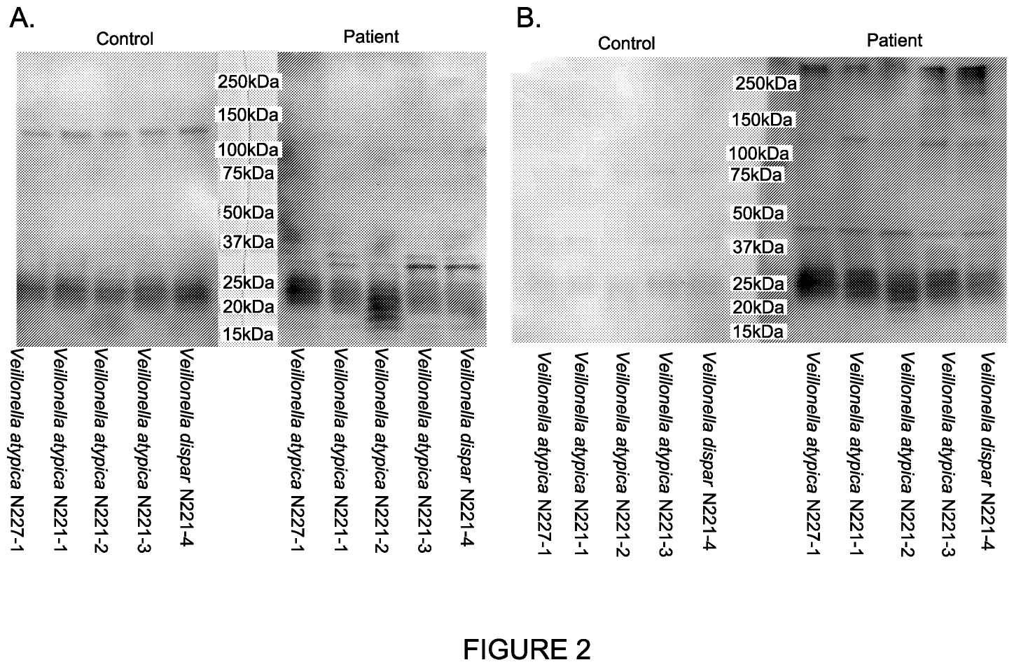

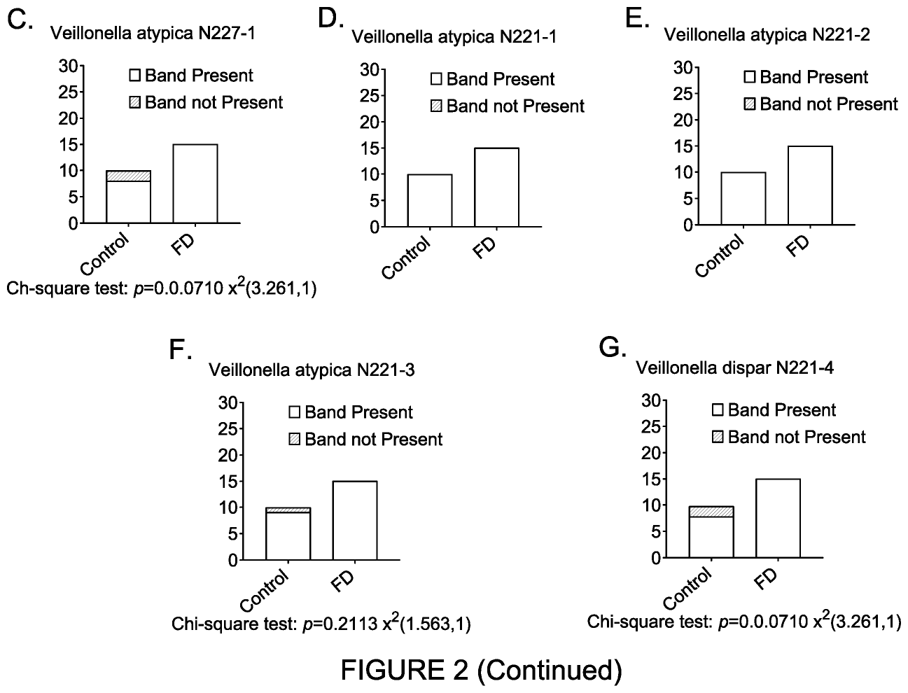

The five strains affiliated with Veillonella spp. selected were screened using the plasma samples from 10 controls and 15 functional dyspepsia patients. The majority of both types of plasma samples interacted with polypeptides of ˜100-150 kDa, ˜37-50 kDa and ˜20-37 kDa from all five Veillonella strains (FIG. 2 A-B).

The plasma from eight controls (80%) and all 15 functional dyspepsia patients (100%) tested were seropositive towards Veillonella atypica N227-1 (FIG. 2C), and Veillonella dispar N221-4 polypeptides (FIG. 2G) (χ²(3.261, 1) p=0.071). The immunoblots also showed that the plasma from all 10 controls and 15 functional dyspepsia subjects was seroreactive towards polypeptides from Veillonella atypica N221-1 (FIG. 2D), and Veillonella atypica N221-2 (FIG. 2E). Plasma from nine controls (90%) and 15 (100%) functional dyspepsia patients was sero-positive towards polypeptides from Veillonella atypica N221-3 (χ²(1.563,1), p=0.211) (FIG. 2F). Given there was no significant relationship between the presence of antibodies against any of the five Veillonella spp. tested, the presence of this interaction is not considered specific to functional dyspepsia.

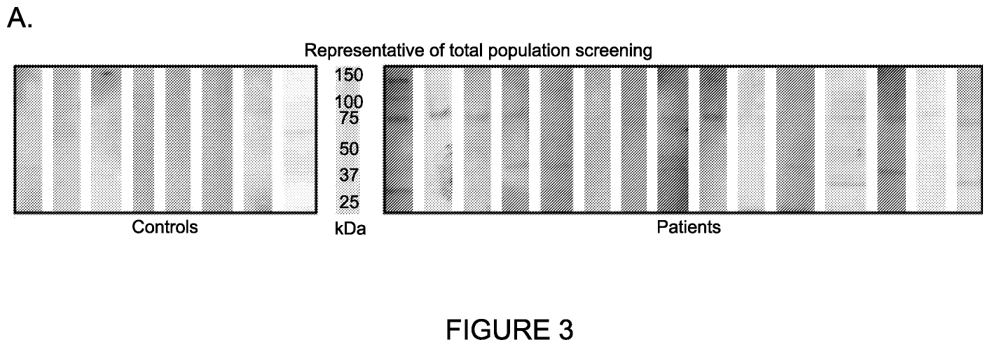

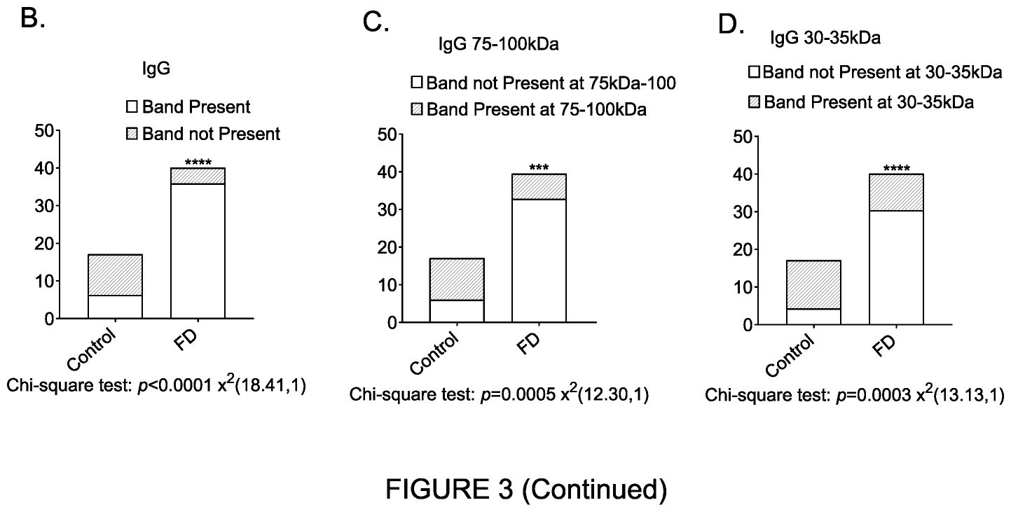

Given the visual intensity of banding representing an IgG specific interaction between Streptococcus salivarius AGIRA0003 and plasma from functional dyspepsia patients on screening, the inventors proceeded to examine the Seroreactivity of a larger cohort of plasma samples towards this bacterium. Forty (40) functional dyspepsia patients were screened against 17 control patients, and representative results of these immunoblots are included in FIG. 3A. In total, 36 (90.00%) functional dyspepsia patients demonstrated a seroreactive response at any molecular weight, compared to 6 (35.29%) controls (χ²(18.41,1), p<0.0001) (FIG. 3B). Within this cohort, 33 (82.50%) functional dyspepsia patients had a banding pattern located between 100-75 kDa, in contrast to 6 (35.29%) of the controls (χ²(12.30, 1),p=0.0005) (FIG. 3C). There was a banding pattern between 30-35 kDa detected in 30 (75.00%) of the functional dyspepsia cohort, compared to 4 (23.53%) of the controls (χ²(13.13, 1), p=0.0003) (FIG. 3D). The statistical analysis of this novel interaction validates that there is a significant association between the production of IgG antibodies directed against specific proteins of Streptococcus salivarius AGIRA0003 and a diagnosis of functional dyspepsia according to the Rome criteria.

### Example 4—Absence of Relationship Between the Presence or Absence of IgM, IgA or IgE Antibodies Directed Against Streptococcus salivarius AGIRA0003 and Functional Dyspepsia

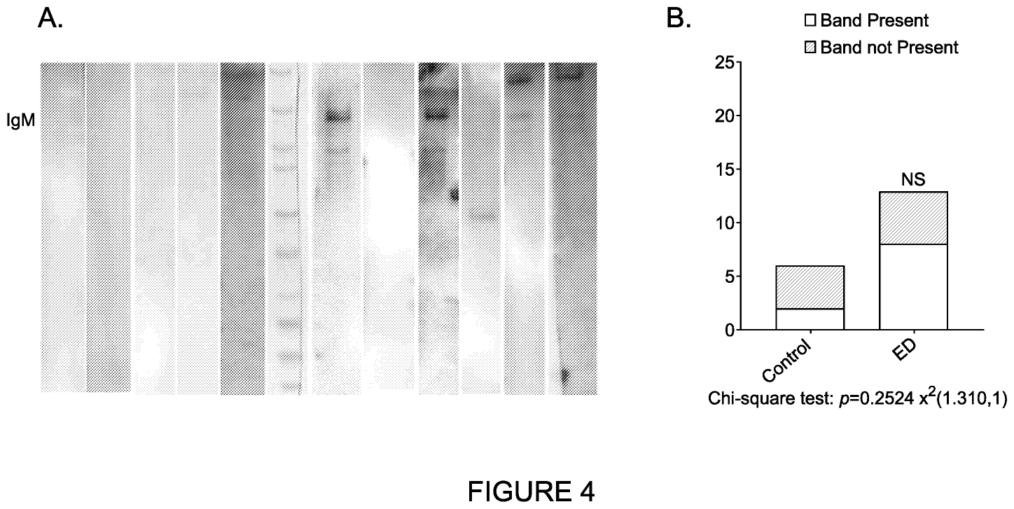

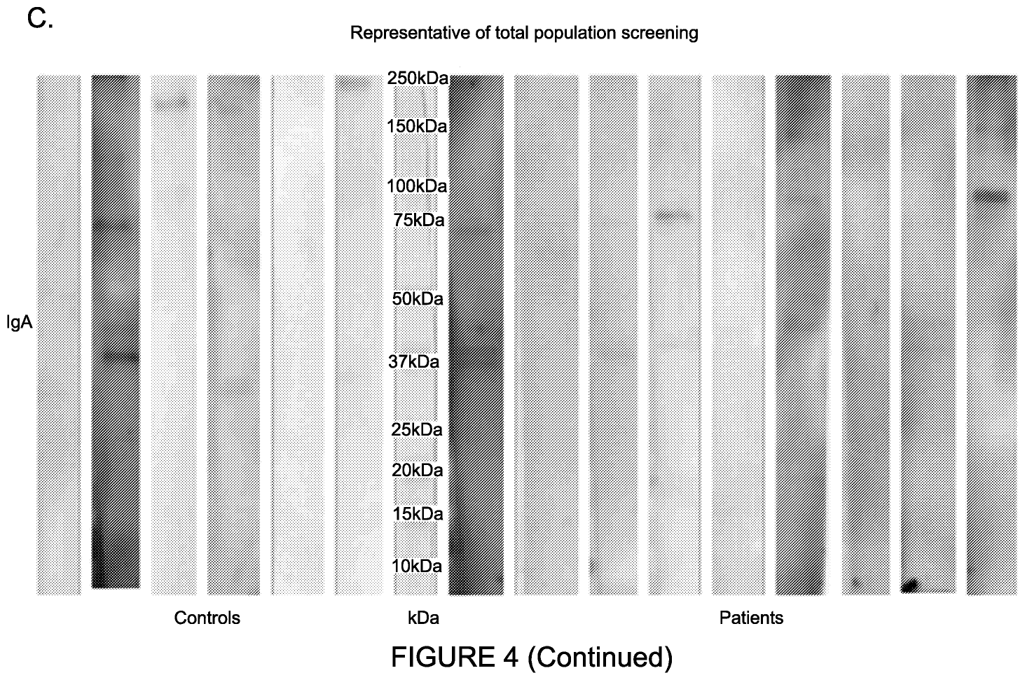

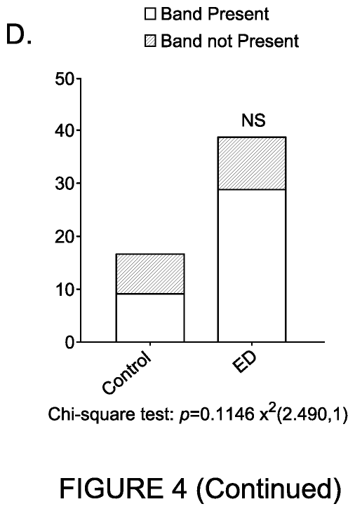

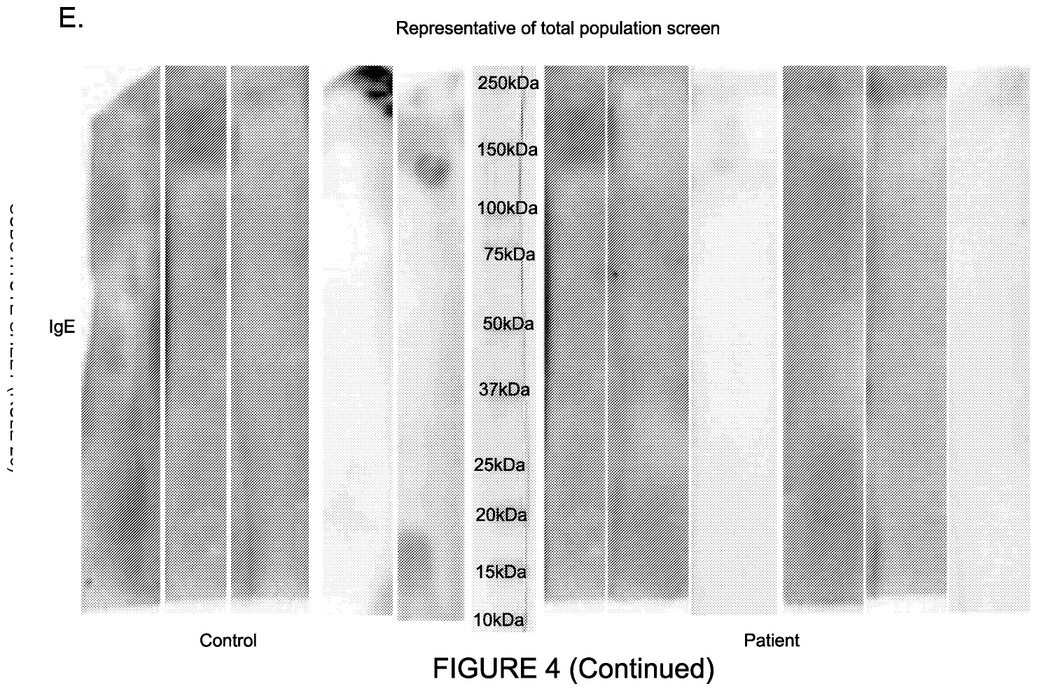

Given the presence of IgG antibodies in the plasma of functional dyspepsia patients, the inventors screened the same Streptococcus salivarius AGIRA0003 isolates against human plasma using IgM, IgA and IgE secondary antibodies to determine if immunoglobulins of these classes could also be detected against the mucosal microbial species. Probing of 6 controls and 13 functional dyspepsia patients with IgM (FIG. 4 A) identified interactions with bacterial proteins between 50-200 kDa in 2 (33.33%) controls and 8 (61.54%) of patients which were not of significance (FIG. 4B). There was no significant relationship between IgA seroreactivity against Streptococcus salivarius AGIRA0003 and functional dyspepsia status (FIG. 4C), which was present in 9 (52.94%) of the 17 controls screened, compared to 29 (74.36%) of the 39 functional dyspepsia patients included (FIG. 4D). Screening of 8 controls and 21 functional dyspepsia patients with IgE antibodies failed to identify any banding at any molecular weight in either cohort (FIG. 4E).

### Example 5—IgG Antibodies Against Streptococcus salivarius AGIRA0003 are not Associated with Potential Confounding Factors in Functional Dyspepsia Patients

To ensure the relationship between an IgG response against Streptococcus salivarius AGIRA0003 and functional dyspepsia was not the result of a confounding characteristic of these patients, the functional dyspepsia cohort was divided by those who had a detectable IgG response (functional dyspepsia IgG+ve, seropositive) and those who did not (functional dyspepsia IgG-ve, seronegative). Correlation analysis was performed for both the 70-100 kDa bands (Table 2) and 30-35 kDa (Table 3) bands separately. There was no statistical difference between functional dyspepsia patients positive or negative for the 70-100 kDa band, or the 30-35 kDa band with regards to Helicobactor pylori status, IBS as a co-morbidity, or PPI, H2RA, or NSAIDs usage.

TABLE 2
POTENTIAL CORRELATIONS BETWEEN SEROREACTIVITY TO STREPTOCOCCUS
SALIVARIUS AGIRA0003 70-100 KDA AND DEMOGRAPHIC INFORMATION

| | FD IgG −ve at 70-100 kDa | FD IgG +ve at 70-100 kDa | p value |
|---|---|---|---|
| H. pylori infection (%) | 0/7 (0.00) | 1/21 (4.55) | >0.9999 |
| IBS co-morbidity (%) | 2/7 (28.57) | 12/27 (44.44) | 0.6722 |
| PPI usage (%) | 3/5 (60.00) | 10/23 (43.48) | 0.6389 |
| H2RA usage (%) | 1/5 (20.00) | 3/23 (13.04) | >0.9999 |
| NSAIDs usage (%) | 1/5 (20.00) | 2/23 (8.70) | 0.4594 |

Fisher's exact test. Data not available for all included samples, /n for each category.

TABLE 3
POTENTIAL CORRELATIONS BETWEEN SEROREACTIVITY TO STREPTOCOCCUS
SALIVARIUS AGIRA0003 30-35 KDA AND DEMOGRAPHIC INFORMATION

| | FD IgG −ve at 30-35 kDa | FD IgG +ve at 30-35 kDa | p value |
|---|---|---|---|
| H. pylori infection (%) | 0/7 (0.00) | 1/21 (4.76) | >0.9999 |
| IBS co-morbidity (%) | 3/9 (33.33) | 11/25 (44.00) | 0.7041 |
| PPI usage (%) | 1/6 (16.67) | 12/22 (54.55) | 0.1727 |
| H2RA usage (%) | 1/6 (16.67) | 3/22 (13.64) | >0.9999 |
| NSAIDs usage (%) | 0/6 (0.00) | 3/22 (13.64) | >0.9999 |

Fisher's exact test. Data not available for all included samples, /n for each category.

### Example 6—Associations of IgG Antibodies Against Streptococcus salivarius AGIRA0003 and Immune Features of Functional Dyspepsia

Given the association of duodenal eosinophilia and increased peripheral T cells expressing the α4 β7 integrins and chemokine-receptor 9 (CCR9) associated with gut-homing in functional dyspepsia, the inventors investigated if these features were associated with the presence or absence of IgG antibodies directed against Streptococcus salivarius AGIRA0003.

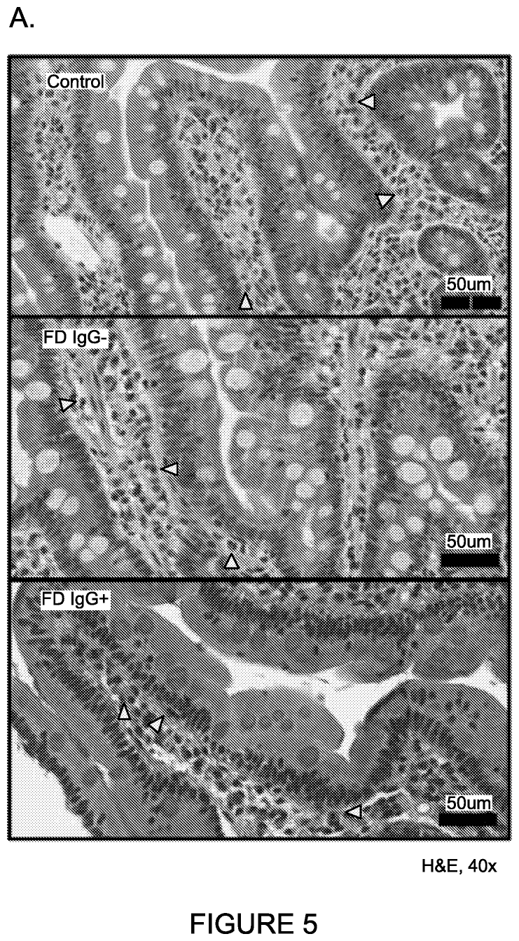

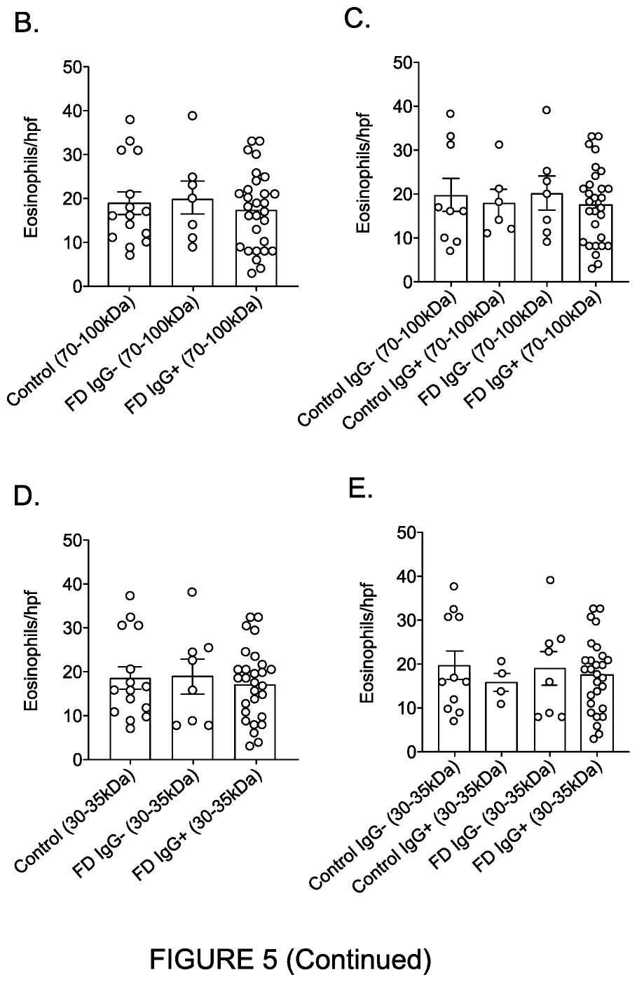

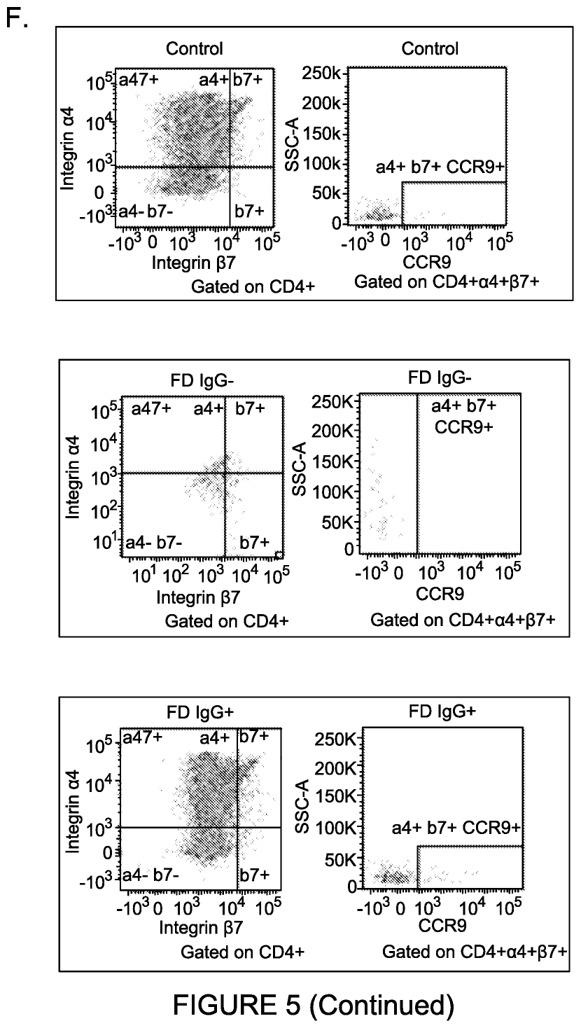

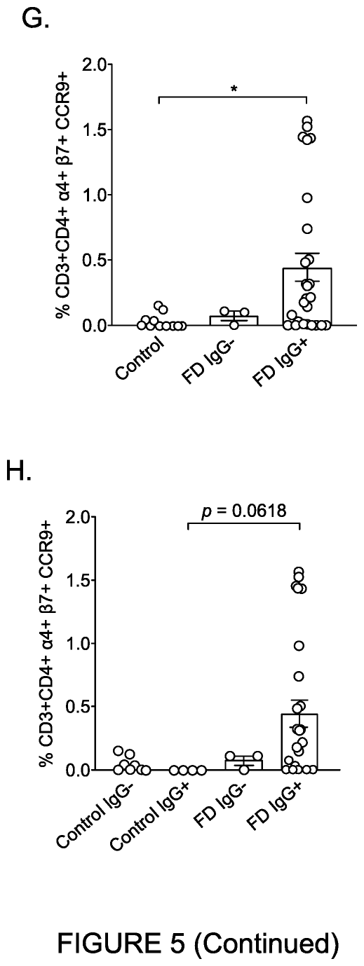

Duodenal biopsies were unavailable for 2 of the control participants and 4 functional dyspepsia patients included in this study, and as such, are not included in this analysis. There was no association between duodenal eosinophil count (FIG. 5A) and IgG seroreactive status in controls, sero-positive or sero-negative FD patients at 70-100 kDa (p>0.9999 for all comparisons) (FIG. 5B). The same result was seen for the 30-35 kDa molecular weight seroreactive band (p>0.9999 for all comparisons) (FIG. 5C). Further stratification of controls based on seroreactive status also revealed no difference in duodenal eosinophil number and seroreactivity status between FD patients and controls at 70-100 kDa (p>0.9999 for all comparisons) (FIG. 5D). Similarly, no relationship was found between duodenal eosinophil number and seroreactivity between controls and FD patients at 30-35 kDa when controls were divided by seroreactive status (p>0.9999 for all comparisons) (FIG. 5E).

When examined by flow cytometry (FIG. 5F), IgG+FD patients had a significantly higher proportion of CD3+CD4+α4+β7+CCR9+cells, compared to controls (0.02925±0.0522 vs 0.4415±0.5599, p=0.014) (FIG. 5G). There was no difference between IgG-patients (0.0700±0.06083) and the control cohorts or the IgG+cohort (p>0.9999 for both comparisons). When the control population in this dataset was separated into IgG-(0.0439 □0.05957) and IgG+(0.0000±0.0000), the proportion of CD3+CD4+α4+β7+CCR9+cells was unchanged between IgG-controls and IgG+FD patients (p=0.062) (FIG. 5H). There was no difference between the IgG+FD cohort and the IgG-FD cohort (p>0.9999) or the IgG-control cohort (p>0.9999). These data suggests an association between the presence of a seroreactive response to S. salivarius AGIRA0003 and increased proportions of gut-homing T cells in FD patients when compared to controls. There is no difference between the proportion of gut-homing T cells and seroreactive status in the control population.

### Example 7—Identification of the Antigenic Proteins from Streptococcus salivarius AGIRA0003

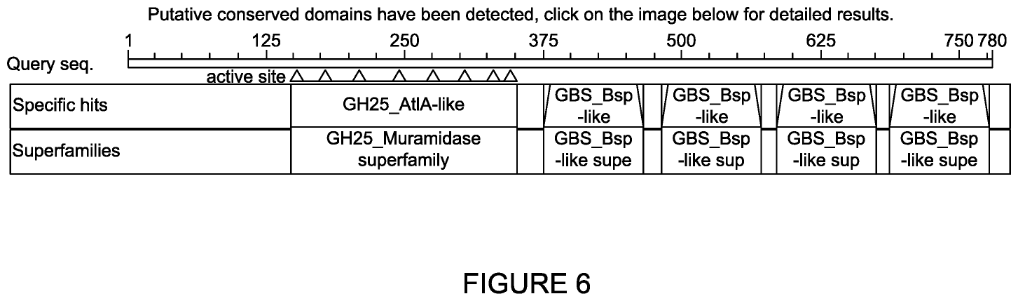

Given the specificity of the interaction between patient plasma and proteins separated from a lysate of Streptococcus salivarius AGIRA0003, the inventors used mass spectrometry to define the amino acid sequence of tryptic peptide fragments produced from the seroreactive polypeptides extracted from the immunoblots. These data were then manually compared to the CDS predicted from the draft genome of S. salivarius AGIRA0003. These comparisons identified multiple tryptic peptides derived from the excised protein at ˜70-100 kDa (i.e. (GB_1D_03_B1) as being derived from a presumptive “Group B Streptococcus (GBS) B Surface Protein (Bsp)-like repeat protein” (ExPASy bioinformatics resource portal theoretical molecular weight=85.04115 kDa, theoretical pI=6.49) (FIG. 6). In addition, multiple tryptic peptides produced from the polypeptide at ˜30-35 kDa (i.e. GB_1D_03_B2) were mapped to the CDS for the S. salivarius AGIRA0003 30S ribosomal protein S2 (ExPASy bioinformatics resource portal theoretical molecular weight=28.35238 kDa, theoretical pI=5.04) (Table 4). The inventors have therefore identified the antigenic proteins of S. salivarius AGIRA0003 as GBS Bsp-repeat domain protein (75-100 kDa; SEQ ID NO:1), implicated in bacterial colonisation, binding of bacterial cells to host epithelial cells and interactions between bacterial cells, and the 30s ribosomal subunit S2 protein (30-35 kDa; SEQ ID NO:2).

TABLE 4
PROTEIN SEQUENCES FOR IDENTIFIED SEROREACTIVE PROTEINS IN STREPTOCOCCUS
SALIVARIUS AGIRA0003 CLINICAL ISOLATES

| Protein | Sequence |
|---|---|
| PROKKA_00585 | MRTKDFIYYASAAVLLAVTTQVAQADEVATTKTPSVTEENQYQSATAAEIFGG |
| GBS Bsp-like | EAALPVTPKSTVSAPAATSEVAKASAPAVSMSPASQSSEAATASTSVTSSVVSSE |
| repeat protein | SATASTSATSSETSNSAVATPAKLTNSTDVPSQTLKVQPKTFIDVSSHNGDISVD |
| (SEQ ID NO: 1) | DYRALARQGVGGVVVKLTEDTWYNNPKAPSQVRNAQIAGLQVSTYHFSRYT |
| | TEEEARAEARFYIQAAQKLNLPKSTVMVNDFEDSNMLPNINRNTQAWVNEM |
| | RKHGYNNLMFYTSASWLDENNLGYRGPVSTSQFGIENFWVAQYPSSSLTATSA |
| | KNMRYNAKTGAWQFSATANLLPGKHVFDQSVDYTGRFTANASVEADPTQGDL |
| | SGTISIVNNNPTLGSFDVVISNVKAPNGVQTVSVPIWSEINGQDDIIWYTANRQN |
| | NGTYTVNVKASAHKNSTGLYNVHLYYVQKDGQLTGVGGTTTQVFIGKKPEISV |
| | FANLSISKNENNGTFTIIAKNLKGLEGYKEVKIPFWSHANGMSDIKWYTPTRQ |
| | ADGSYTVTVKASDHENANGRYEAQVFYIDARGQKRFVQKAFVERNDPSKPTG |
| | VISITNNKDSGTFDVVISDVYSPKGVRTVQVPIWSEVDGQDDIRWYEATRQTDG |
| | NYKVTVQVANHKYSTGIYNVHLYYIQNDGSQIGVGGTQTNVTLSEPKADLAIT |
| | GLNNATGSYDVVISNLVAPRGFKEVLVPTWSEKNGQDDIIWYKAAKQANGDY |
| | KVTVRSSNHKGDSGLYNSHVYLVDNDGKFIGLGGKSVTLKRA |
| PROKKA_01557 | MAVISMKQLLEAGVHFGHQTRRWNPKMAKYIFTERNGIHVIDLQQTVKMAD |
| 30S ribosomal | TAYEFVREAAANDAVILFVGTKKQAAEAVAEEATRAGQYYINHRWLGGTL |
| protein S2 (SEQ | TNWDTIQKRIARLKEIKQMEADGTFEVLPKKEVALLNKQRARLEKFLGGIED |
| ID NO: 2) | MPRIPDVIYIVDPHKEQIAVKEAKKLGIPVVAMVDTNADPDEIDVIIPANDDAIR |
| | AVKLITSKMADAIIEGNQGEDASADFQEAAAADSIEEIVEVVEGDNN |

Bold represents tryptic peptide masses obtain from mass spectrometry data and where they match the coding sequence in the S. salivarius AGIRA0003 open reading frames. Underlined amino acid sequences represent distinct peptides identified by mass spectrometry analysis that are derived from the larger tryptic fragment.

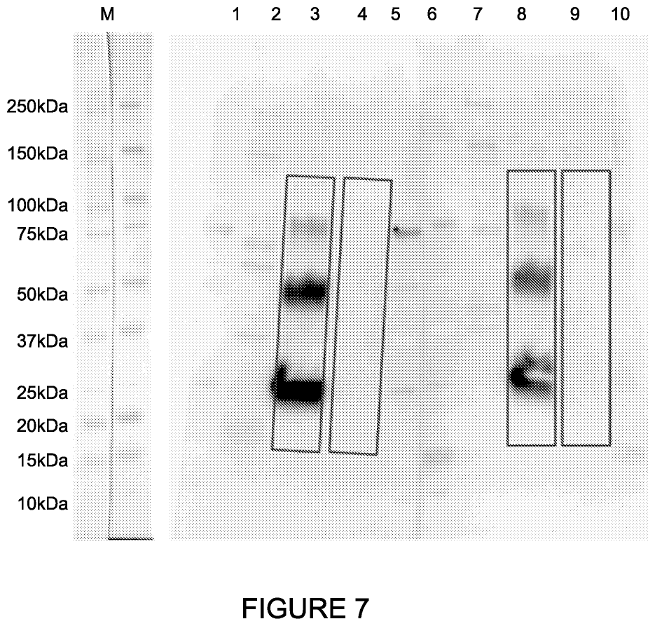

To further characterise the antigenic proteins, the inventors have used an IgG pulldown assay to isolate total IgG antibodies from patient plasma, incubating the resulting sample with the S. salivarius AGIRA0003 total protein lysate to bind the specific antigenic proteins to specific patient antibodies. The seroreactive proteins at the corresponding molecular weight positions were extracted from a gel (FIG. 7) and also provided matched pull down elution products for mass spectrometric analysis.

### Example 8—Sensitivity and Specificity of Seroreactivity and Functional Dyspepsia

As shown in Table 5 below, the presence of an IgG seroreactive response at any molecular weight was associated with functional dyspepsia with a sensitivity of 90% (95% CI: 0.7695-0.9604) and specificity of 64.71% (95% CI: 0.4130-0.8269). For a seroreactive response at 70-100 kDa, the sensitivity was 82.50% (95% CI: 0.6805-0.9125) and specificity was 64.71% (95% CI: 0.4130-0.8269). For a seroreactive response at 30-35 kDa, the sensitivity was 75.00% (95% CI: 0.5981-0.8581) and specificity was 76.47% (95% CI: 0.5274-0.9044). The IgG seroreactive response at any molecular weight was associated with functional dyspepsia with a positive predictive value (PPV) of 85.71% (95% CI: 0.7216-0.9328), a negative predictive value (NPV) of 73.33% (95% CI: 0.4805-0.8910) and a likelihood ratio of 2.550. A seroreactive response at 70-100 kDa was associated with functional dyspepsia with a positive predictive value (PPV) of 84.62% (95% CI: 0.7027-0.9275), a negative predictive value (NPV) of 61.11% (95% CI: 0.3862-0.7969) and a likelihood ratio of 2.338. For seroreactive responses at 30-35 kDa, the association with functional dyspepsia had a positive predictive value (PPV) of 88.24% (95% CI: 0.7338-0.9533), a negative predictive value (NPV) of 56.52% (95% CI: 0.3681-0.7437) and a likelihood ratio of 3.188.

TABLE 5
SENSITIVITY AND SPECIFICITY FOR IGG ANTIBODIES AGAINST
STREPTOCOCCUS SALIVARIUS AGIRA0003 AS A MARKER OF FD.

| | | IgG seroreactivity | IgG seroreactivity |
|---|---|---|---|
| | IgG seroreactivity | (70-100 kDa only) | (330-35 kDa only) |
| Sensitivity (95% CI) | 90% (0.770-0.960) | 82.50% (0.681-0.913) | 75.00% (0.598-0.858) |
| Specificity (95% CI) | 64.71% (0.413-0.827) | 64.71% (0.413-0.827) | 76.47% (0.527-0.904) |
| Positive predictive value | 85.71% (0.722-0.933) | 84.62% (0.703-0.928) | 88.24% (0.734-0.953) |
| (95% CI) | | | |
| Negative predictive | 73.33% (0.481-0.891) | 61.11% (0.386-0.797) | 56.52% (0.368-0.744) |
| value (95% CI) | | | |
| Likelihood ratio | 2.550 | 2.338 | 3.188 |

Wilson/Brown method for 95% confidence interval (CI).

### Example 9—Streptococcus salivarius Strain AGIRA0003 Genome Analysis

The inventors have described the complete genome of Streptococcus salivarius strain AGIRA0003 in Hoedt et al. 2021, Microbiology Resource Announcements (https://doi.org/10.1128/MRA0075-21).

The inventors have used a bioinformatics approach to further characterise the capacity of AGIRA0003 to elicit immune responses in comparison to other S. salivarius strains. The pan genome of representative S. salivarius strains was generated and searched to determine the presence, number, and type of the virulence genes in each genome. Additionally, the genome files of nucleotide sequence for S. salivarius strains ATCC 7073, M18, K12, and AGIRA0003 were uploaded to RAST (Rapid Annotation using Subsystem Technology). Here the annotation service and SEED viewer report were used to examine the subsystems related to Virulence, Disease and Defence. This was completed to confirm and identify any virulence genes missed in the initial literature search for these strains. Using this in silico approach the inventors identified a unique “virulence” gene dually annotated as a flagella motor protein (see Table 6) and Lysobacter enzymogenes type III secretion system cytoplasmic ring protein SctQ. The type III secretion system has been described in the literature as a structure that provide gram-negative pathogens with a unique virulence mechanism, facilitating the injection of bacterial effector proteins directly into the host cell cytoplasm, bypassing the extracellular milieu.

TABLE 6
NUMBER AND TYPE OF VIRULENCE GENES IN EACH STREPTOCOCCUS SALIVARIUS
STRAIN, IDENTIFIED THROUGH RAST SEED VIEWER AND EDGAR

| | | | Probiotic | |
|---|---|---|---|---|
| >no of genes | | Probiotic | M18 + | |
| Virulence Factor | AGIRA0003 | K12 + plasmid | plasmid | ATCC_7073 |
| Adhesion | 0 | 1 | 0 | 3 |
| Urease | 7 | 8 | 7 | 0 |
| Capsule | 1 | 1 | 1 | 1 |
| Flagellum | 3 | 3 | 3 | 3 |
| Flagella motor protein FLIM/FLIN | 1 | 0 | 0 | 0 |
| Haemolysin | 1 | 1 | 1 | 1 |
| LPXTG-anchored Surface Proteins | 16 | 17 | 16 | 19 |
| Siderophore | 4 | 2 | 4 | 2 |
| Lipotechoic acid | 5 | 5 | 5 | 5 |
| Superoxide dismutase | 1 | 1 | 1 | 1 |
| Collagen-binding protein | 1 | 0 | 1 | 1 |
| Glucosyltransferases | 27 | 26 | 32 | 24 |
| Autolysin | 4 | 5 | 5 | 4 |
| Choline binding protein A | 1 | 0 | 1 | 1 |
| IgA1 protease | 1 | 1 | 1 | 1 |
| Pilus | 1 | 3 | 3 | 2 |
| Enolase (FBP) | 1 | 1 | 1 | 1 |
| GAPDH (FBP) | 1 | 1 | 1 | 1 |
| C5a pepsidase | 1 | 0 | 1 | 1 |
| Resistance to antibiotics and toxic | 4 | 9 | 7 | 8 |
| compounds | | | | |
| Colbalt/zinc/cadmium resistance | 4 | 8 | 4 | 7 |
| Mercuric reductase/ mercury | 3 | 3 | 3 | 3 |
| resistance | | | | |
| Streptococcus pneumoniae | 5 | 4 | 5 | 5 |
| Vancomycin Tolerance Locus | | | | |
| Resistance to fluoroquinolones | 2 | 2 | 2 | 2 |
| Multidrug Resistance Efflux Pumps | 1 | 0 | 1 | 1 |
| Mycobacterium virulence operon | 9 | 9 | 9 | 9 |
| RAST identified genes: | 28 | 35 | 31 | 35 |
| SUM Total | 105 | 111 | 115 | 106 |

1. A method for diagnosing a functional gastrointestinal disorder in a subject, the method comprising detecting IgG antibodies in a sample obtained from the subject, wherein the IgG antibodies recognise one or more antigens from Streptococcus salivarius.

2. A method according to claim 1, wherein the functional gastrointestinal disorder is functional dyspepsia or irritable bowel syndrome.

3. A method according to claim 2, wherein the functional gastrointestinal disorder is functional dyspepsia.

4. A method according to any one of claims 1 to 3, wherein the sample is a blood sample.

5. A method according to claim 4, wherein the sample is a plasma sample.

6. A method according to any one of claims 1 to 5, wherein the Streptococcus salivarius is S. salivarius AGIRA0003, deposited pursuant to the Budapest Treaty, National Measurement Institute (NMI), Australia on 28 Apr. 2021 under Accession Number V21/008005.

7. A method according to any one of claims 1 to 5, wherein the Streptococcus salivarius is S. salivarius AGIRA0003 and the one or more antigens are selected from Group B Streptococcal (GBS) Bsp-like repeat protein, 30S ribosomal protein S2, and antigenic peptides or fragments thereof.

8. A method according to any one of claims 1 to 7, wherein the IgG antibodies are detected using an enzyme-linked immunosorbent assay (ELISA).

9. A method according to any one of claims 1 to 8, wherein the method comprises contacting the sample with GBS Bsp-like repeat protein or an antigenic or immunoreactive peptide or fragment thereof, and/or 30S ribosomal protein S2 or an antigenic or immunoreactive peptide or fragment thereof.

10. A method according to any one of claims 1 to 9, wherein the method comprises comparing a determined level of the IgG antibodies in the sample to a reference or control value, wherein the reference of control value represents the level of the IgG antibodies in the absence of a functional gastrointestinal disorder.

11. A method according to claim 10, wherein the reference or control value is derived from one or more individuals known not to have a functional gastrointestinal disorder.

12. A kit for detecting IgG antibodies for use in the diagnosis of a functional gastrointestinal disorder, the kit comprising one or more antigenic or immunoreactive proteins, peptides or fragments selected from GBS Bsp-like repeat protein or an antigenic or immunoreactive peptide or fragment thereof, and/or 30S ribosomal protein S2 or an antigenic or immunoreactive peptide or fragment thereof, wherein said IgG antibodies recognise one or more antigens from Streptococcus salivarius.

13. A method for selecting a subject for treatment for a functional gastrointestinal disorder, comprising:
i) executing the step of detecting IgG antibodies in samples obtained from one or more subjects, and optionally determining the level of said antibodies to thereby diagnose the functional gastrointestinal disorder, wherein said IgG antibodies recognise one or more antigens from Streptococcus salivarius; and
ii) selecting a subject for treatment for the functional gastrointestinal disorder based on said IgG antibody detection or levels.

14. A method for monitoring the response of a subject to a treatment regime for a functional gastrointestinal disorder, or for evaluating the efficacy of a treatment regime in a subject with a functional gastrointestinal disorder, the method comprising:
i) treating the subject with a treatment regime for the functional gastrointestinal disorder for a period sufficient to evaluate the efficacy of the regime;
ii) obtaining a sample from the subject and detecting IgG antibodies in the sample, optionally determining the level of said antibodies, wherein said IgG antibodies recognise one or more antigens from Streptococcus salivarius;
iii) repeating step ii) at least once over a period of time while the subject is administered said treatment regime; and
iv) determining whether the level of said IgG antibodies change over the period of time,
to thereby determine the response of the subject to the treatment regime and/or the efficacy of the treatment regime.


SEQUENCE LISTING (2 sequences)

SEQ ID NO: 1 - protein, 784 aa, Streptococcus salivarius
MRTKDFIYYASAAVLLAVTTQVAQADEVATTKTPSVTEENQYQSATAAEIFGGEAALPVTPKSTVSAPAATSEVAKASAPAVSMSPASQSSEAATASTSVTSSVVSSESATASTSATSSETSNSAVATPAKLTNSTDVPSQTLKVQPKTFIDVSSHNGDISVDDYRALARQGVGGVVVKLTEDTWYNNPKAPSQVRNAQIAGLQVSTYHFSRYTTEEEARAEARFYIQAAQKLNLPKSTVMVNDFEDSNMLPNINRNTQAWVNEMRKHGYNNLMFYTSASWLDENNLGYRGPVSTSQFGIENFWVAQYPSSSLTATSAKNMRYNAKTGAWQFSATANLLPGKHVFDQSVDYTGRFTANASVEADPTQGDLSGTISIVNNNPTLGSFDVVISNVKAPNGVQTVSVPIWSEINGQDDIIWYTANRQNNGTYTVNVKASAHKNSTGLYNVHLYYVQKDGQLTGVGGTTTQVFIGKKPEISVFANLSISKNENNGTFTIIAKNLKGLEGYKEVKIPFWSHANGMSDIKWYTPTRQADGSYTVTVKASDHENANGRYEAQVFYIDARGQKRFVQKAFVERNDPSKPTGVISITNNKDSGTFDVVISDVYSPKGVRTVQVPIWSEVDGQDDIRWYEATRQTDGNYKVTVQVANHKYSTGIYNVHLYYIQNDGSQIGVGGTQTNVTLSEPKADLAITGLNNATGSYDVVISNLVAPRGFKEVLVPTWSEKNGQDDIIWYKAAKQANGDYKVTVRSSNHKGDSGLYNSHVYLVDNDGKFIGLGGKSVTLKRA

SEQ ID NO: 2 - protein, 255 aa, Streptococcus salivarius
MAVISMKQLLEAGVHFGHQTRRWNPKMAKYIFTERNGIHVIDLQQTVKMADTAYEFVREAAANDAVILFVGTKKQAAEAVAEEATRAGQYYINHRWLGGTLTNWDTIQKRIARLKEIKQMEADGTFEVLPKKEVALLNKQRARLEKFLGGIEDMPRIPDVIYIVDPHKEQIAVKEAKKLGIPVVAMVDTNADPDEIDVIIPANDDAIRAVKLITSKMADAIIEGNQGEDASADFQEAAAADSIEEIVEVVEGDNN